(19) United States
(12) Patent Application Publication
(10) Pub. No.: US 2022/0325267 A1
(43) Pub. Date: Oct. 13, 2022

(54) CONTROLLING MULTIPLE HETEROGENOUS MAGNETIC BACTERIA AT A SOLID-LIQUID INTERFACE USING UNIFORM MAGNETIC FIELDS

(71) Applicant: Ohio State Innovation Foundation, Columbus, OH (US)

(72) Inventor: Wijesinghe Mudiyanselage Hiran Shanaka Wijesinghe, Old Bridge, NJ (US)

(21) Appl. No.: 17/667,058

(22) Filed: Feb. 8, 2022

Related U.S. Application Data
(60) Provisional application No. 63/147,279, filed on Feb. 9, 2021.

(51) Int. Cl. C12N 13/00; H01F 1/42

(52) U.S. Cl. CPC C12N 13/00; H01F 1/42

(57) ABSTRACT

Flagellated magnetotactic bacteria (MTB), specifically AMB-1 bacteria, are provided as a system of microrobots, and the heterogeneity of their hydrodynamic interactions with a solid-liquid boundary wall is systematically exploited to control multiple microrobots using a global magnetic field. A method comprises providing a plurality of a microrobots and controlling the microrobots using a global magnetic field.

### CROSS-REFERENCE TO RELATED APPLICATIONS

This application claims the benefit of U.S. provisional patent application No. 63/147,279, filed on Feb. 9, 2021, and entitled “METHOD FOR CONTROLLING MULTIPLE HETEROGENOUS MAGNETIC BACTERIA AT A SOLID-LIQUID INTERFACE USING UNIFORM MAGNETIC FIELDS,” the disclosure of which is expressly incorporated herein by reference in its entirety.

### STATEMENT OF GOVERNMENT SUPPORT

This invention was made with government support under 1424138 and 1710598 awarded by the National Science Foundation. The government has certain rights in the invention.

### BACKGROUND

Manipulatable miniaturized robots have a variety of applications in biomedical sciences and manufacturing. Microrobots capable of navigating in vivo blood vessels have the potential to revolutionize non-invasive surgery and targeted drug delivery for cancer and other disease treatment. Drug discovery can be assisted by legions of microrobots designed to manipulate biological cells in vitro. For these applications to be feasible, however, one needs to be able to handle more than one miniature microrobot simultaneously. This is challenging for several reasons. Extremely small robots do not allow for on-board computation and navigation, and complex propulsion mechanisms. Robots can be designed such that they are controllable remotely. However, they need to be independently controllable from a remote source.

Remote-controlled microrobots have been proposed that use various actuation principles, e.g., optical tweezer, chemical, ultrasound, electrostatic, and magnetic approaches. Specifically, magnetic fields are considered safer for biological cells and tissues. Artificial, bio-hybrid, and biological magnetic microswimmers have also been proposed for remote-controlled applications using external magnetic fields. Among them, magnetotactic bacteria (MTB) such as AMB-1 (strain) are specially convenient since they have well-adapted swimming mechanisms for low-Reynolds number environments, and are easier to produce in large quantities.

However, the inability to independently control a team of MTB using global fields has posed a considerable challenge in building a microrobotic system. Previous work has utilized heterogeneity in the robots to simultaneously control several microrobots. Exploiting heterogeneity in static friction, no-slip wall conditions, dimensions, resonant frequencies, and magnetic moments have found limited success in controlling several microrobots using global magnetic fields.

It is with respect to these and other considerations that the various aspects and embodiments of the present disclosure are presented.

### SUMMARY

Flagellated magnetotactic bacteria (MTB), specifically AMB-1 bacteria, are provided as a system of microrobots, and the heterogeneity of their hydrodynamic interactions with a solid-liquid boundary wall is systematically exploited to control multiple microrobots using a global magnetic field.

In some implementations, a method comprises: providing a plurality of a microrobots; and controlling the microrobots using a global magnetic field. As used herein, the term global refers to the fact that all microrobots are subjected to the same magnetic field, as opposed to local magnetic fields applied on individual microrobots separately.

Implementations may include some or all of the following features. The microrobots are magnetotactic bacteria (MTB). The MTB are dispersed in a dilute suspension in a microchannel. The MTB comprise AMB-1 bacteria. The heterogeneity of the hydrodynamic interactions of the microrobots with a solid-liquid boundary wall is systematically exploited to control the microrobots using the global magnetic field. The microrobots are magnetic beads. The method further comprises applying the global magnetic field to the microrobots to align the swimming axis of each of the microrobots with the magnetic field, to obtain a distribution of swimming velocities of the microrobots near a surface subjected to an external magnetic field. The method further comprises mapping the swimming velocities onto a single multidimensional Euclidean space, and determining a basis system of magnetic fields that sufficiently span a target configuration of the bacteria. The global magnetic field is time varying and uniform. The microrobots are spaced far enough apart so that they do not interact with each other.

In some implementations, a system comprises: a plurality of microrobots; and a magnetic field configured to control the microrobots.

Implementations may include some or all of the following features. The microrobots are magnetotactic bacteria (MTB). The MTB are dispersed in a dilute suspension in a microchannel. The MTB comprise AMB-1 bacteria. The magnetic field is a global magnetic field, and the heterogeneity of the hydrodynamic interactions of the microrobots with a solid-liquid boundary wall is systematically exploited to control the microrobots using the global magnetic field. The microrobots are magnetic beads. The system further comprises a computer configured to apply the magnetic field to the microrobots to align the swimming axis of each of the microrobots with the magnetic field, to obtain a distribution of swimming velocities of the microrobots near a surface subjected to an external magnetic field. The computer is further configured to map the swimming velocities onto a single multidimensional Euclidean space, and determine a basis system of magnetic fields that sufficiently span a target configuration of the microrobots. The magnetic field is time varying and uniform. The microrobots are spaced far enough apart so that they do not interact with each other.

In some implementations, a system comprises: a plurality of microrobots; a microchannel with a dilute suspension of the microrobots; a plurality of electromagnets; and a computer configured to: control the electromagnets; check whether there are sufficient control signals in order to drive the microrobots toward their targets; when sufficient control signals are not yet available, the computer maps new control signals to response vectors using a computer vision system and the electromagnets; and once sufficient control signals are identified, the computer solves for the time vector τ, plans a path in which order to apply the control signals; and executes the planned path by activating uniform magnetic fields.

Implementations may include some or all of the following features. Planning the path comprises restricting the microrobots to a camera field of view and/or avoiding two or more bacteria from colliding or coming too close to one another. The microrobots are magnetotactic bacteria (MTB). The MTB are dispersed in a dilute suspension in a microchannel. The MTB comprise AMB-1 bacteria. The microrobots are magnetic beads. The computer is further configured to apply a magnetic field to the microrobots to align the swimming axis of each of the microrobots with the magnetic field, to obtain a distribution of swimming velocities of the microrobots near a surface subjected to an external magnetic field. The computer is further configured to map the swimming velocities onto a single multidimensional Euclidean space, and determine a basis system of magnetic fields that sufficiently span a target configuration of the bacteria. The magnetic field is time varying and uniform. The microrobots are spaced far enough apart so that they do not interact with each other.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIGS. 1A and 1B are diagrams that show a magnetically aligned bacterium swimming near a surface subject to magnetic fields;

FIG. 2 is a diagram of an implementation of a real-time magnetotactic bacteria (MTB) manipulator;

FIGS. 3A and 3B are diagrams helpful to explain control signals and paths of a bacterium;

FIG. 4 is a diagram of an implementation of a system of controlling magnetic bacteria using magnetic fields;

FIG. 5 is an operational flow of an implementation of a method of controlling magnetic bacteria using magnetic fields; and

FIG. 6 shows an exemplary computing environment in which example embodiments and aspects may be implemented.

### DETAILED DESCRIPTION

This description provides examples not intended to limit the scope of the appended claims. The figures generally indicate the features of the examples, where it is understood and appreciated that like reference numerals are used to refer to like elements. Reference in the specification to “one embodiment” or “an embodiment” or “an example embodiment” means that a particular feature, structure, or characteristic described is included in at least one embodiment described herein and does not imply that the feature, structure, or characteristic is present in all embodiments described herein.

In order to gain simultaneous control over the positions of magnetotactic bacteria (MTB) relative to the underlying surface, the systems and methods described herein exploit the variations of multiple properties of MTB within the population. For instance, even though the typical length of an AMB-1 cell body is ˜3 μm, there exist a distribution of body lengths in any given AMB-1 population. Similarly, the helical shapes of the cell bodies are not identical, and the thrust force each swimming cell generates is different. There are many such properties that, when combined, bestow unique hydrodynamic characteristics to a bacterium swimming near a surface. The systems and methods described herein amplify these small differences between bacteria using external magnetic fields, and use them to independently control the cells on the surface. Several advantages over the existing methods and techniques are obtained. Bacteria are typically easier to culture in large quantities compared to fabricating remote controllable microrobots capable of swimming. Cultured bacteria inherently have heterogeneities in their characteristics. The systems and methods described herein exploit a combination of such heterogeneities rather than relying on a single property. This makes it easier to find sufficient heterogeneity in a given system of bacteria. Furthermore, implementations can be provided as a “lab-on-a-chip” microfluidic device in a small form factor.

When a magnetic field is applied to a motile MTB, its swimming axis aligns with the magnetic field. However, the trajectory of an MTB swimming near a surface is also dependent on the hydrodynamic interactions with the wall. As a result of the inhomogeneity of various properties of the swimming bacteria in a population, a distribution of swimming velocities can be observed in a system of MTB near a surface subjected to an external magnetic field. The proposed method maps the swimming velocities of many MTB onto a single multidimensional Euclidean space, and seeks a basis system of magnetic fields that sufficiently span a target configuration of the bacteria. The proposal incorporates a real-time particle tracking computer vision system together with a computerized three-dimensional (3D) electromagnet setup in order to control multiple bacteria simultaneously. How the basis system of magnetic fields (“control signals”) is utilized to control multiple bacteria is explained further herein.

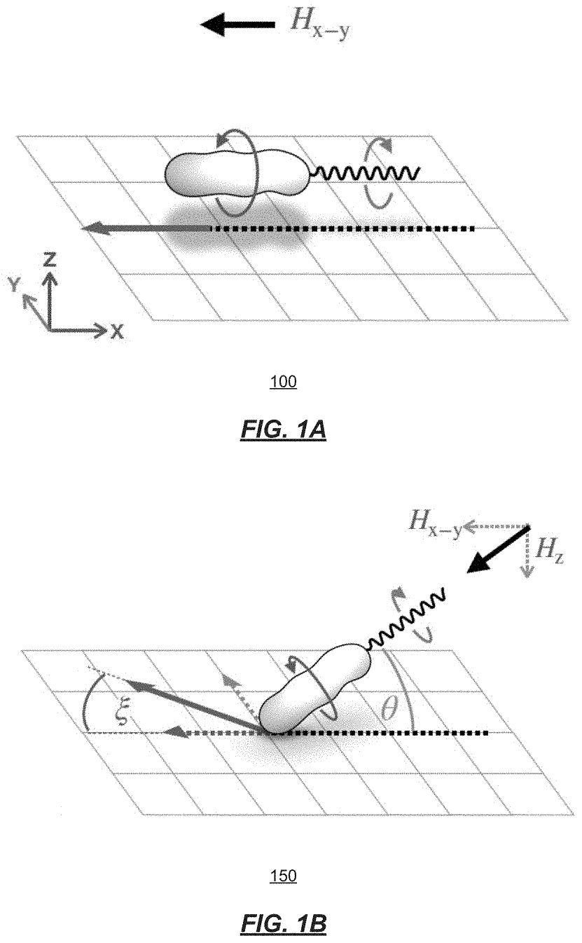

Consider an MTB swimming near a surface while being aligned with an external magnetic field as shown in the diagrams 100, 150 respectively of FIGS. 1A and 1B. Its velocity on the x-y plane is determined by the tilt angle θ. When the bacterium is parallel to the surface, the swimming direction is aligned with the projection of the swimming axis onto the x-y plane (dotted line), and the speed is determined by the swimming velocity of the bacterium. However, when the bacterium is tilted, the “forward” swimming velocity is decreased, and an additional velocity component in the y direction emerges due to the hydrodynamic interaction of the cell body with the surface. This can be understood as a “rolling” effect of the cell body due to its rotation and the drag mismatch between the upper and lower portions of the cell body surface. When the cell body is parallel to the surface, such a rolling effect is not observed irrespective of the distance between the cell and the surface. This is attributed to the matching drag torques on the counter-rotating cell body and the flagellum at equilibrium.

More particularly, in FIGS. 1A and 1B, a magnetically aligned AMB-1 bacterium near a surface swims in a direction (solid arrows on the x-y plane) determined by the intrinsic properties of the bacterium, and the ‘tilt angle’ θ relative to the surface. In FIG. 1A, a bacterium oriented parallel to the surface using a magnetic field H_{x-y} swims in the direction of the long axis of the cell body. In FIG. 1B, when the same bacterium is tilted by an angle θ relative to the surface (‘tilt angle’), the bacterium veers off its swimming axis by an angle ξ (‘veer angle’) on the x-y plane. Here, the curved arrows indicate the rotation of the cell body and the flagellum respectively. A dotted arrow indicates a ‘rolling’ velocity of the cell body on the x-y plane due to its rotation that leads to the veer angle.

Suppose that a “control signal” W represents a specific uniform magnetic field applied on the suspension. ψ entails all properties that uniquely defines the control signal. For example, once ψ is specified, the strength of the magnetic field, the out-of-plane (z) component of the magnetic field, the in-plane component, and the clockwise angle ζ of the in-plane field relative to the x direction are all specified by ψ.

Once the control signal ψ is applied on the system of MTB, each bacterium responds by moving in the x-y plane at a bacterium-specific velocity. Assuming the suspension is sufficiently dilute so that there are no hydrodynamic interactions between bacteria, the response of the entire system per unit time is represented by a vector Ξ.

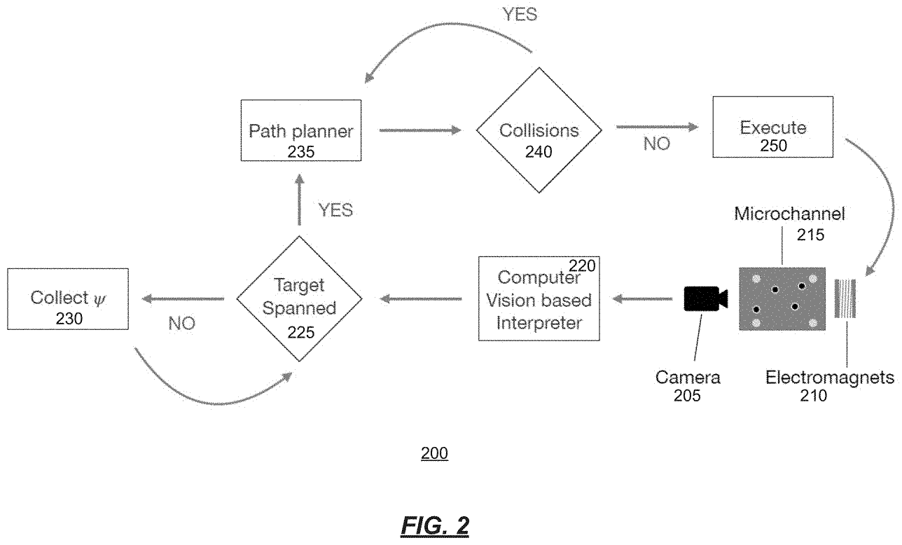

FIG. 2 is a diagram of a layout of a real-time MTB manipulator. The system 200 comprises a camera 205 and an electromagnetic apparatus (electromagnets 210) connected to a computer (not shown). The computer reads the camera 205 feed and locates the bacteria, checks if it “knows” sufficient control signals and collects more control signals, plans a path until collisions can be avoided, and executes the path. Magnetic control, camera feedback and path planning should occur in a near real-time feedback loop (ideally, only limited by the camera frame rate). The electromagnets 210, by applying current thereto for example, generate a uniform 3D magnetic field.

The algorithm presented here is intended to be incorporated into a setup comprising a microchannel 215 with a dilute suspension of MTB, a microscope and a digital camera 205, a computer, and computerized electromagnets 210. The computer should be able to handle the camera feed and control the electromagnets. As summarized in FIG. 2, the computer should interpret the camera feed and locate the bacteria on the microchannel floor (a “computer vision” system shown as computer vision based interpreter 220).

Given a target set of locations for the bacteria, the computer checks whether it has sufficient control signals in order to drive the bacteria toward their targets. If sufficient control signals are not yet available, the computer maps new control signals (at 230) to response vectors using the computer vision system, and the electromagnets. Once sufficient control signals are identified (the target is spanned at 225), the computer solves for the time vector r and proceeds to plan a path with the path planner 235 (i.e., in which order to apply the control signals). Path planning entails steps such as restricting the bacteria to the camera field of view, avoiding two or more bacteria from colliding or coming too close (unwanted hydrodynamic interactions between bacteria should be avoided) at 240. Then, the planned path is executed at 250 by activating uniform magnetic fields accordingly. The computer may use an objective function to bring all bacteria “close enough” to the target.

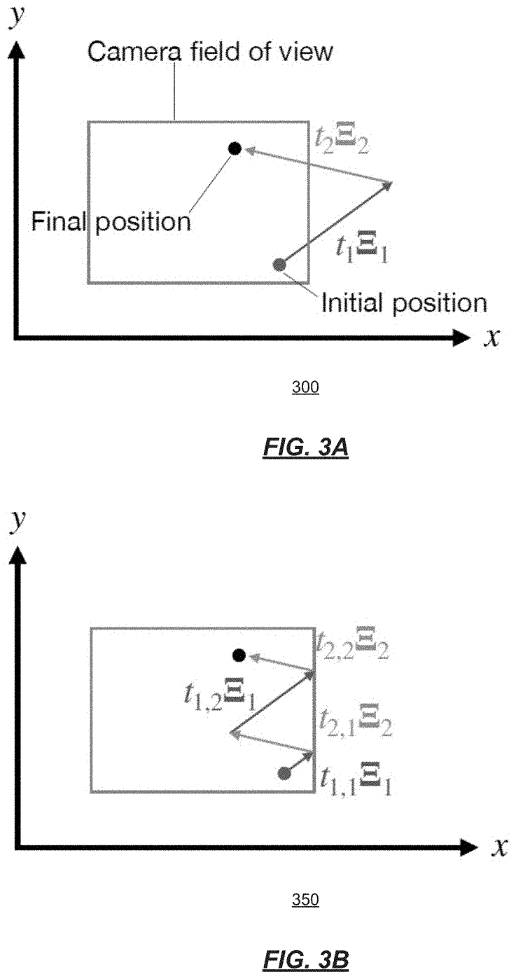

FIGS. 3A and 3B show diagrams 300, 350, respectively, that show restriction of MTB to the camera field of view. In FIG. 3A, given a set of control signals ψi and the corresponding response vectors Ξi, an individual bacterium may swim outside the field of view due to the order in which the control signals are applied. In FIG. 3B, a different path can be planned in which the control signals are applied in an alternative order. Signals can even be broken into smaller pieces and rearranged to make sure MTB stay in the camera frame. There is no unique solution for the specific trajectory, and usually there are infinitely many possibilities that can keep the bacterium in the frame. The same idea presented in FIGS. 3A and 3B can be extended to N bacteria by recognizing that in the 2N dimensional tΞ-space, the camera field of view is generalized to a hyperrectangle. In this case, the point in the tΞ-space representing the positions of all bacteria should be kept inside the hyperrectangle using a rearrangement of the control signals similarly to FIG. 3B.

More particularly, because the computer should be able to track MTB on the microchannel without interruptions, it is important to keep all MTB within the camera frame, especially when magnetic fields are applied, in which case bacteria can potentially swim outside the frame. Consider a system of N=1 bacterium. Suppose that a target is specified, and the known control signals are sufficient to drive the bacterium to the target, using the solution to the time vector T. If the application of ψ1 for time t1, and then ψ2 for time t2, etc. takes the bacterium outside the camera frame before bringing it back, one can apply the same control signals piece-wise with an altered sequence to make sure the bacterium stays in the frame. This same idea can be extended to N bacteria using the tΞ-space representation. x and y coordinates of all N bacteria are represented by a single point in the ta-space (tΞ-space position vector). The camera field of view generalizes to a 2N-dimensional hyperrectangle (a generalization of a rectangle to higher dimensions). An altered sequence of the control signals should be used to keep the ta-space position vector inside this hyperrectangle (FIGS. 3A and 3B).

The target locations of all bacteria can be represented by a single point P_{target} in the tΞ-space. Similarly, at any given time, the x-y plane positions of all N bacteria are represented by another position P_{current} in the tΞ-space. A distance measure between these two points can provide an objective function. If the objective function is ‘sufficiently minimized’, i.e., the distance between the target and the current position is below a predetermined value, the computer can stop attempting to drive cells further closer to the target. One suggestion is to use the Euclidean distance between P_{target} and P_{current} as the objective function.

Hydrodynamic interactions between bacteria can occur that can adversely interfere with the response vectors. A situation where bacteria come too close can be recognized by examining the tΞ-space position vector. A path planner could be programmed to avoid such regions in the tΞ-space where at least two bacteria overlap or come too close, say, less than 5 μm.

Another consideration is the existence of sharp turns. Bacteria take a finite amount of time to reorient when a magnetic field is applied. This time can be reduced if sharp turns are avoided. A path planner may be programmed to ease bacteria into new orientations.

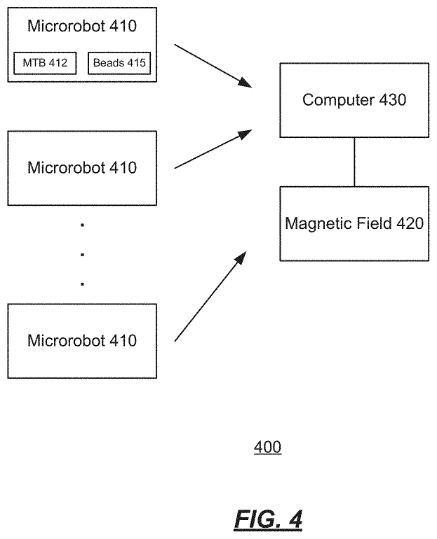

FIG. 4 is a diagram of an implementation of a system 400 of controlling magnetic bacteria using magnetic fields. The system 400 comprises a plurality of microrobots 410 and a magnetic field 420 configured to control the microrobots 410. The microrobots 410 are MTB 412 and/or magnetic beads 415, depending on the implementation. The microrobots 410 are spaced far enough apart so that they do not interact with each other. In an implementation, the MTB 412 are dispersed in a dilute suspension in a microchannel. In an implementation, the MTB 412 comprise AMB-1 bacteria. In an implementation, the magnetic field 420 is time varying and uniform.

In an implementation, the magnetic field 420 is a global magnetic field, and a heterogeneity of hydrodynamic interactions of the microrobots 410 with a solid-liquid boundary wall is used to control the microrobots 410 using the global magnetic field.

The system 400 further comprises a computer 430 configured to apply the magnetic field 420 to the microrobots 410 to align the swimming axis of each of the microrobots 410 with the magnetic field 420, to obtain a distribution of swimming velocities of the microrobots 410 near a surface subjected to an external magnetic field.

In an implementation, the computer 430 (also referred to herein as a computing device) is configured to map the swimming velocities onto a single multidimensional Euclidean space, and determine a basis system of magnetic fields that sufficiently span a target configuration of the bacteria.

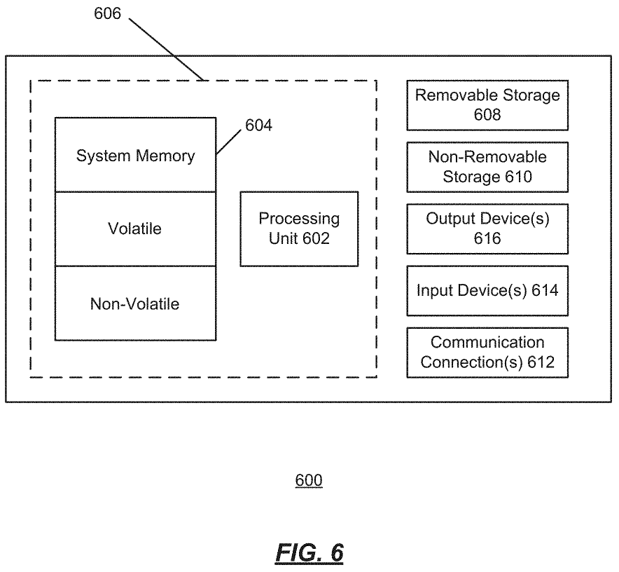

The computer (computing device) 430 may be implemented using a variety of computing devices such as desktop computers, laptop computers, tablets, etc. Other types of computing devices may be supported. A suitable computing device is illustrated in FIG. 6 as the computing device 600.

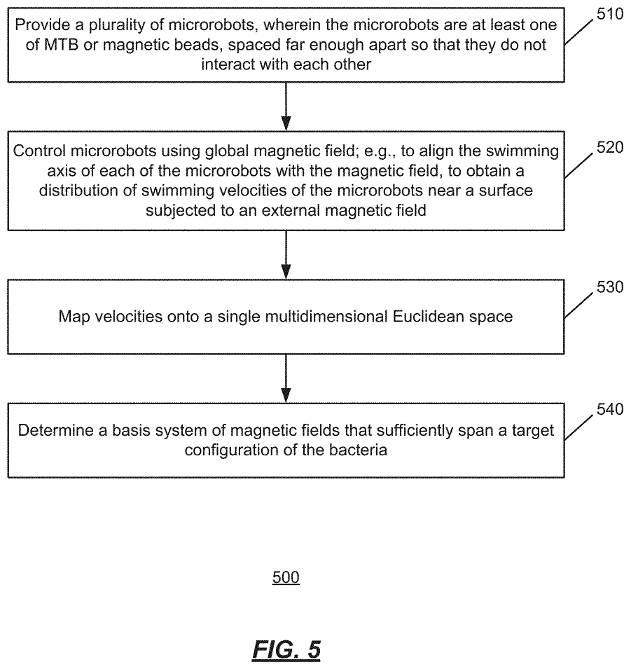

FIG. 5 is an operational flow of an implementation of a method 500 of controlling magnetic bacteria using magnetic fields.

At 510, a plurality of a microrobots is provided, wherein the microrobots are at least one of MTB or magnetic beads. The microrobots are spaced far enough apart so that they do not interact with each other. In an implementation, the MTB are dispersed in a dilute suspension in a microchannel. The MTB may comprise AMB-1 bacteria in an implementation.

At 520, the microrobots are controlled using a global magnetic field. In an implementation, a heterogeneity of hydrodynamic interactions of the microrobots with a solid-liquid boundary wall is used to control the microrobots using a global magnetic field. The global magnetic field may be time varying and uniform.

In some implementations, a global magnetic field is applied to the microrobots to align the swimming axis of each of the microrobots with the magnetic field, to obtain a distribution of swimming velocities of the microrobots near a surface subjected to an external magnetic field.

At 530, the swimming velocities are mapped onto a single multidimensional Euclidean space.

At 540, a basis system of magnetic fields that sufficiently span a target configuration of the bacteria is determined.

Thus, systems and methods are described herein that control a system of MTB near a surface using static magnetic fields applied equally to all bacteria simultaneously. It was experimentally observed that AMB-1 bacteria differ in their response to certain magnetic fields. Specifically, their swimming speed and veer angles were observed to be different from cell to cell. Subsequently, a mathematical model was developed that systematically amplifies these small differences in order to independently control multiple bacteria using uniform magnetic fields.

By harnessing the intra-population variations of responses to control signals, the signal-response framework described herein paves the way for new applications that require targeted control of multiple micron-sized particles. Potential applications include novel “lab-on-a-chip” devices and micro-fabrication systems, target delivery systems at micron scale, and on-the-fly reconfigurable micro-mixing and micro-pumping devices.

The systems and methods described herein provide several advantages over the existing systems and methods. The “robots” utilized here are magnetotactic bacteria, which are biocompatible and biodegradable. It is relatively easier to produce MTB in large quantities compared to fabricating artificial microrobots. Moreover, because the heterogeneity of the hydrodynamic wall interactions of MTB originate from a variety of sources (e.g., cell body length and shape, flagellar lengths, flagellar motor torques, magnetic moment, no-slip wall conditions, etc.) rather than a single heterogeneous property, a wider distribution of the heterogeneous property can be expected. Such a wider distribution makes it easier to control more microrobots simultaneously.

FIG. 6 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 6, an exemplary system for implementing aspects described herein includes a computing device, such as computing device 600. In its most basic configuration, computing device 600 typically includes at least one processing unit 602 and memory 604. Depending on the exact configuration and type of computing device, memory 604 may be volatile (such as random access memory (RAM)), non-volatile (such as read-only memory (ROM), flash memory, etc.), or some combination of the two. This most basic configuration is illustrated in FIG. 6 by dashed line 606.

Computing device 600 may have additional features/functionality. For example, computing device 600 may include additional storage (removable and/or non-removable) including, but not limited to, magnetic or optical disks or tape. Such additional storage is illustrated in FIG. 6 by removable storage 608 and non-removable storage 610.

Computing device 600 typically includes a variety of computer readable media. Computer readable media can be any available media that can be accessed by the device 600 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 604, removable storage 608, and non-removable storage 610 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 600. Any such computer storage media may be part of computing device 600.

Computing device 600 may contain communication connection(s) 612 that allow the device to communicate with other devices. Computing device 600 may also have input device(s) 614 such as a keyboard, mouse, pen, voice input device, touch input device, etc. Output device(s) 616 such as a display, speakers, printer, etc. may also be included. All these devices are well known in the art and need not be discussed at length here.

As used herein, the singular form “a,” “an,” and “the” include plural references unless the context clearly dictates otherwise. As used herein, the terms “can,” “may,” “optionally,” “can optionally,” and “may optionally” are used interchangeably and are meant to include cases in which the condition occurs as well as cases in which the condition does not occur.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method comprising:
providing a plurality of a microrobots, wherein the microrobots are at least one of magnetotactic bacteria (MTB) or magnetic beads, wherein the microrobots are spaced far enough apart so that they do not interact with each other; and
controlling the microrobots using a global magnetic field.

2. The method of claim 1, wherein the MTB are dispersed in a dilute suspension in a microchannel.

3. The method of claim 1, wherein the MTB comprise AMB-1 bacteria.

4. The method of claim 1, wherein a heterogeneity of hydrodynamic interactions of the microrobots with a solid-liquid boundary wall is used to control the microrobots using a global magnetic field.

5. The method of claim 4, further comprising applying the global magnetic field to the microrobots to align the swimming axis of each of the microrobots with the magnetic field, to obtain a distribution of swimming velocities of the microrobots near a surface subjected to an external magnetic field.

6. The method of claim 5, further comprising mapping the swimming velocities onto a single multidimensional Euclidean space, and determining a basis system of magnetic fields that sufficiently span a target configuration of the bacteria.

7. The method of claim 4, wherein the global magnetic field is time varying and uniform.

8. A system comprising:
a plurality of microrobots, wherein the microrobots are at least one of magnetotactic bacteria (MTB) or magnetic beads, wherein the microrobots are spaced far enough apart so that they do not interact with each other; and
a magnetic field configured to control the microrobots.

9. The system of claim 8, wherein the MTB are dispersed in a dilute suspension in a microchannel.

10. The system of claim 8, wherein the MTB comprise AMB-1 bacteria.

11. The system of claim 8, wherein the magnetic field is a global magnetic field, and a heterogeneity of hydrodynamic interactions of the microrobots with a solid-liquid boundary wall is used to control the microrobots using the global magnetic field.

12. The system of claim 8, further comprising a computer configured to apply the magnetic field to the microrobots to align the swimming axis of each of the microrobots with the magnetic field, to obtain a distribution of swimming velocities of the microrobots near a surface subjected to an external magnetic field.

13. The system of claim 12, wherein the computer is further configured to map the swimming velocities onto a single multidimensional Euclidean space, and determine a basis system of magnetic fields that sufficiently span a target configuration of the bacteria.

14. The system of claim 8, wherein the magnetic field is time varying and uniform.

15. A system comprising:
a plurality of microrobots, wherein the microrobots are at least one of magnetotactic bacteria (MTB) or magnetic beads, wherein the microrobots are spaced far enough apart so that they do not interact with each other;
a microchannel with a dilute suspension of the microrobots,
a plurality of electromagnets; and
a computer configured to:
control the electromagnets;
check whether there are sufficient control signals in order to drive the microrobots toward their targets;
when sufficient control signals are not yet available, the computer maps new control signals to response vectors using a computer vision system and the electromagnets; and
once sufficient control signals are identified, the computer solves for the time vector τ, plans a path in which order to apply the control signals; and executes the planned path by activating uniform magnetic fields.

16. The system of claim 15, wherein planning the path comprises at least one of restricting the microrobots to a camera field of view or avoiding two or more bacteria from colliding or coming too close to one another.

17. The system of claim 15, wherein the MTB are dispersed in a dilute suspension in a microchannel, and wherein the magnetic field is time varying and uniform.

18. The system of claim 15, wherein the MTB comprise AMB-1 bacteria.

19. The system of claim 15, wherein the computer is further configured to apply a magnetic field to the microrobots to align the swimming axis of each of the microrobots with the magnetic field, to obtain a distribution of swimming velocities of the microrobots near a surface subjected to an external magnetic field.

20. The system of claim 19, wherein the computer is further configured to map the swimming velocities onto a single multidimensional Euclidean space, and determine a basis system of magnetic fields that sufficiently span a target configuration of the bacteria.
